(19) United States
(12) Patent Application Publication
(10) Pub. No.: US 2015/0366458 A1
(43) Pub. Date: Dec. 24, 2015

(54) APPARATUS AND METHOD FOR FREQUENCY-DOMAIN THERMO-ACOUSTIC TOMOGRAPHIC IMAGING

(71) Applicants: Stephan Kellnberger, Kunzing (DE); Vasilis Ntziachristos, Grafelfing (DE); George Sergiadis, Peraia (GR); Nikolaos Deliolanis, Stuttgart (DE); Murad Omar, Munchen (DE)

(72) Inventors: Stephan Kellnberger, Kunzing (DE); Vasilis Ntziachristos, Grafelfing (DE); George Sergiadis, Peraia (GR); Nikolaos Deliolanis, Stuttgart (DE); Murad Omar, Munchen (DE)

(73) Assignee: Helmholtz Zentrum Munchen Deutsches Forschungszentrum Fur Gesundheit Und Umwelt (GmbH), Neuherberg (DE)

(21) Appl. No.: 14/399,272

(22) PCT Filed: May 7, 2012
(86) PCT No.: PCT/EP2012/001959

§ 371 Date: Jan. 22, 2015

(51) Int. Cl. A61B 5/00; A61B 19/00; G01N 21/17

(52) U.S. Cl. CPC A61B 5/0095; G01N 21/1702; A61B 19/54; A61B 5/6852; G01N 2021/1706; G01N 2201/06113; G01N 2201/08; G01N 2201/12

(57) ABSTRACT

An imaging apparatus (100), configured for thermoacoustic tomographic imaging a region of interest (2) in an object (1), comprises a source device (10) being arranged for emitting an electromagnetic energy input into the region of interest (2), a detector device (20) being arranged for detecting mechanical wave response signals generated in the region of interest (2) along multiple angular projection directions in response to the electromagnetic energy input, and an image data acquisition and processing device (30) being arranged for providing tomographic image data representing the image of the region of interest (2) on the basis of the mechanical wave response signals, wherein the source device (10) is adapted for continuously emitting the electromagnetic energy input with a predetermined input modulation, and the image data acquisition and processing device (30) is adapted for converting the mechanical wave response signals into the frequency domain and for performing data processing and image reconstruction in the frequency domain or in the time domain. Furthermore, an imaging method for thermoacoustic tomographic imaging a region of interest (2) in an object (1) is described.

### TECHNICAL FIELD

The present invention relates to an imaging apparatus and an imaging method for thermoacoustic (including opto-acoustic and photoacoustic) tomographic imaging a region of interest in an object. In particular, the inventive technique is capable of generating cross-sectional images of biological tissue through detection of electromagnetic absorption across the electromagnetic spectrum.

### TECHNICAL BACKGROUND OF THE INVENTION

Thermo-acoustic imaging relies on the absorption of electromagnetic energy by tissue and the corresponding tissue heating, resulting in acoustic pressure release because of thermoelastic expansion of the tissue. While the term thermo-acoustic imaging is employed to determine generally any form of energy absorption by tissue, the terms optoacoustic or photo-acoustic more specifically reflect imaging when the energy employed is light. Based on this physical phenomenon, optoacoustic imaging (OAI) and thermo-acoustic imaging (TAI) techniques, or photoacoustic imaging (PAI) and tomography (PAT) as the general term, emerged over the past years. Herein, optoacoustics refers to generation of mechanical pressure waves by light in the visible or near-infra red region whereas thermo-acoustics is employed to cover all bands of the electromagnetic spectrum, including but limited to the radiofrequency and microwave bands.

Thermo-acoustic imaging has been typically implemented by using short high energy pulses of electromagnetic energy, termed herein the time domain (TD). For efficient TD thermo-acoustic signal generation, the pulse duration is generally below 1 μs (e.g. in the order of a few nanoseconds) with pulse energies up to several tens and hundreds of mJoule. The pulse repetition rate of TD optoacoustic/thermo-acoustic systems is in the range of 10 Hz to several hundreds of kHz. Correspondingly, the duty cycle is generally in the order of 0.1% -10⁻⁵%. The time domain approaches favor practical imaging applications providing sufficient signal to noise ratio (SNR) to generate images of tissues of biological interest. Pulsed thermo-acoustics operate upon time of flight measurements, referring locally induced acoustic pressure signals to distance from the detector with the traveling acoustic wave speed-time dependence. In the time domain, generated thermo-acoustic signals typically represent a broadband response to short electromagnetic or optical pulses with the spectral components of the induced signal containing information about the shape and dimension of the absorber.

There are TD optoacoustic imaging approaches which generate tomographic images of tissues and cells, resolving anatomical, functional and molecular features of the tissue investigated. In addition, TD optoacoustic imaging can quantitatively reveal the distribution of tissue biomarkers within tissue, typically facilitated by multispectral illumination of the tissue at several wavelengths following optoacoustic signal analysis using photon propagation models. This time domain multispectral approach is disclosed in US 2011/0306857 A1.

More generally, there are several TD thermo-acoustic imaging approaches which are mainly based on a pulse modulated carrier frequency amplification concept. Popular frequency bands are the high MHz and low GHz region with respect to the low RF absorption of biological tissue in this frequency band. In U.S. Pat. No. 6,567,688, a narrowband pulse modulated microwave radiation source at 3 GHz is considered for thermo-acoustic wave excitation. The pulse durations are set to 0.5 μs. Detection of acoustic waves is facilitated with a single element ultrasonic transducer or a multi-element array. A similar approach is described in U.S. Pat. No. 6,216,025. The electromagnetic source which has a carrier frequency of 434 MHz is pulse modulated to 0.5 μs. Taking the geometric shape of the detector which has the dimensions of a bowl into consideration, the main application of the system is thermoacoustic breast cancer imaging. Apart from the carrier frequency amplification mode, a near-field approach was implemented in US 2011/0040176 A1. Instead of pulse modulated carrier wave electromagnetic sources, this concept assumes broadband excitation with short high energy pulses. Tomography is facilitated either by a rotating single element transducer around the object or arranging a multi-element transducer array at distinct positions around the sample.

All above mentioned time domain thermo-acoustic imaging techniques cover tomographic approaches whether in optoacoustics with pulsed optical excitation or thermo-acoustics with RF/microwave excitation with short electromagnetic pulses. Tomography implies digital cross sectional imaging with data collection over multiple angular projections. Nevertheless, the TD tomographic approaches have limitations in terms of complex pulse sources, measurement speed and high peak intensities of excitation pulses operating at low duty cycles.

In contrast to afore mentioned time domain approaches which utilize short high energy pulses with low duty cycle, thermo-acoustic signals can also be induced in frequency domain (FD), using a modulated continuous wave source for excitation of thermo-acoustic signals. U.S. Pat. No. 4,255,971 was among the first methodology applying the photoacoustic effect in a thermo-acoustic microscope. The source, e.g. a laser, was intensity modulated at a modulation frequency of typically 10 kHz to 20 MHz. Tissue was scanned horizontally on a x-y plane, gathering planar surface and subsurface optoacoustic signals from the tissue, resulting in depth-profilometric images of objects.

Fan and Mandelis (Y. Fan and A. Mandelis, J. Acoust. Soc. Am. 116(6), (2004)) were among the first to demonstrate a FD optoacoustic system for subsurface imaging. The system used of a CW ytterbium fiber laser at 1064 nm. The laser beam was modulated with an acousto-optic modulator, driven by a function generator. Imaging was performed with a frequency sweep and heterodyne modulation with lock-in detection. Similar to U.S. Pat. No. 4,255,971, the system performance was limited to 2D surface scanning of tissues.

US 2005/0234319 A1 discloses a FD photothermoacoustic imaging system based on a heterodyned lock-in detection scheme for depth profilometric biomedical imaging. Similar to the afore mentioned publication (Y. Fan and A. Mandelis, J. Acoust. Soc. Am. 116(6), (2004)), imaging is performed on a lateral (x-y) surface scan of tissues, resulting in depth-profilometric images.

Recently, Telenkov et al. (S. Telenkov, A. Mandelis, B. Lashkari, and M. Forcht, J. Appl. Phys., 105, 102029 (2009)) presented a frequency domain based optoacoustic imaging system, operating with frequency chirps from 1 MHz to 5 MHz. Herein, the sample consisting of chicken breast tissue was scanned in the horizontal plane over a defined area. After detection, the acoustic signals were cross correlated with the stimulation signal to calculate the phase delay and time shift from absorbers relative to the detectors. Imaging performance of this frequency domain optoacoustic imaging scanner was comparably limited to lateral (x y) scans of tissue and did not yield tomographic views of targets.

Preliminary investigations on the thermo-acoustic effect with microwave CW excitation were discussed in (G. Ye, PSTD Method for Thermoacoustic Tomography (TAT) and Related Experimental Investigation, Dissertation, Department of Electrical and Computer Engineering in the Graduate School of Duke University (2009)). The developed system consisted of a microwave generator providing a CW carrier frequency at 407 MHz. The high frequent radiation was down converted with frequency mixers to the intermediate excitation frequency 1 MHz to match the detection bandwidth of the ultrasonic element. The thesis focused on thermo-acoustic response due to low frequent CW excitation and did not have intentions for imaging.

In frequency domain opto-acoustic imaging, using CW light sources, three-dimensional imaging was performed by linear x-y scans over the tissue's surface. This horizontal scan ends up in depth profilometric images of the tissue and does not show a cross-sectional 360° view of targets. Furthermore, the conventional three-dimensional optoacoustic imaging has disadvantages in terms of limited view scanning of targets, low imaging velocity, low spatial resolution and low SNR.

### Objective of the Invention

It is an objective of the invention to provide an improved thermo-acoustic imaging apparatus being capable of avoiding limitations of conventional thermo-acoustic imaging techniques. Furthermore, it is an objective of the invention to provide an improved thermoacoustic imaging method being capable of avoiding limitations of conventional thermoacoustic imaging techniques. In particular, the objective of the invention is to provide an apparatus or method for thermo-acoustic imaging being capable of creating three-dimensional image data of an object under investigation with improved imaging velocity, improved spatial resolution and/or improved SNR not restricted to limited view scans. In Particular, the apparatus or method is to be capable of employing dedicated tomographic reconstruction algorithms. Furthermore, the invention is to be capable of providing new application ranges for apparatus or method for thermo-acoustic imaging. Whereas thermoacoustic is mentioned, opto-acoustic (photo-acoustic) is also implied.

These objectives are solved with devices and methods as defined in the independent claims, resp. Advantageous embodiments and applications of the invention are defined in the dependent claims.

### SUMMARY OF THE INVENTION

According to a first aspect of the invention, an imaging apparatus (imaging scanner) is provided which is configured for thermo-acoustic tomographic imaging of a region of interest (target) in an object. The imaging apparatus comprises a source device, a detector device and an image processing device. The source device is adapted for emitting an electromagnetic energy input, which is capable of exciting mechanical waves in the region of interest (ROI). According to the invention, the source device is a continuously emitting, modulated source device. The source device is adapted for continuously emitting the electromagnetic energy input with a predetermined modulation (input modulation). The detector device is adapted for detecting mechanical wave response signals (or: acoustic pressure waves) generated in the region of interest along multiple spatial directions (or: projection directions, travel directions of mechanical waves) through the ROI in response to the electromagnetic energy input. Thus, the source device and detector devices are adapted for tomographic imaging, i. e. cross sectional imaging of the ROI with a data collection over multiple angular projections. Typically, projections over at least 60° are required for image formation, however the wider the angular coverage, e. g. 180° or preferably 360°, the better the image quality achieved. The image processing device is arranged for providing tomographic image data representing the image of the region of interest on the basis of the mechanical wave response signals originating from electromagnetic energy, in particular light absorption. According to the invention, the image processing device is adapted for converting the detected mechanical wave response signals into the frequency domain and for performing data processing in the frequency domain or alternatively—after preprocessing steps and back-conversion into time domain—in the time domain. In frequency domain, induced pressure is dependent on the waveform pattern of the input modulation. Correspondingly, the detection bandwidth of the detection elements can be chosen according to input specifications.

The continuous emission regarded herein could be without interruptions, or it could be intermittent, for example for purposes of multiplexing of energies of multiple frequencies (wavelengths), multiplexing different imaging modalities in hybrid implementations or differentially obtaining measurements in the presence and absence of radiating energy, for example for subtracting background signals generated in the absence of irradiation. Particularly, continuous emission (without interruptions or intermittent) can also be of time, frequency or phase encoded nature, e.g. allowing for simultaneous multispectral illumination of targets at several wavelengths. A differentiating parameter between TD pulsed systems and FD systems with continuous intermittent emission is that the entire pulse period is used for signal generation in TD detection whereas in FD signals are generated during the time interval that the pulse is on, i.e. signals are detected even if the pulse is prolonged for long time intervals. An associated difference herein therefore is that generally the continuous intermittent emission in FD system can be of any duration whereby the pulses in TD system need to be faster than the speed of sound and their duration is limited by the spatial image resolution desired. Other differentiating factors between TD and FD systems, including the source, detection and image reconstruction technology can be derived from the description below.

According to a second aspect of the invention, an imaging method for thermo-acoustic tomographic imaging a region of interest in an object is provided, which comprises the steps of emitting an electromagnetic energy input into the region of interest with a source device, detecting mechanical wave response signals generated in the region of interest along multiple projection directions in response to the electromagnetic energy input with a detector device, and providing tomographic image data representing the image of the region of interest on the basis of electromagnetic energy absorption with an image processing device. According to the invention, the step of emitting the electromagnetic energy input includes a continuously modulated emission. The source device is continuously emitting the electromagnetic energy input with a predetermined modulation. Furthermore, according to the invention, the step of providing the tomographic image data includes a conversion of the mechanical wave response signals into the frequency domain and performing data processing in the frequency domain. Preferably, the imaging method of the invention is conducted using the imaging apparatus according to the above first aspect of the invention.

Preferably, the spatial distribution of absorbers in the region of interest is reconstructed in frequency domain using a pulse compression method, involving the cross correlation from an input modulation signal with the mechanical wave response signals.

Advantageously, alternative imaging and tomographic reconstruction methods can furthermore employ wave solutions using diffracting sources and subsequently invert a corresponding matrix describing the geometrical and operational parameters of the illumination and detection process, the so called weight-matrix corresponding to a forward problem (Avinash C. Kak and Malcolm Slaney, Principles of Computerized Tomographic Imaging—Chapter 6 Tomographic Imaging with Diffracting Sources, IEEE Press (1988)).

The inventive method for frequency domain tomographic imaging comprises consecutive steps of illuminating the ROI with continuous wave radiation, e. g. in the optical or RF/microwave regime of the electromagnetic spectrum, detection of acoustic pressure signals following absorption of optical energy or electromagnetic radiation with at least one detection element and reconstruction of a cross-sectional, tomographic image representing an absorption map of electromagnetic energy. In the case of optical excitation, the resulting tomographic image is a representative map of optical absorption within the target, for RF/microwave excitation, the reconstructed cross-sectional image corresponds to the RF/microwave energy deposition within the imaged target.

Agents can be also employed to modify (increase or decrease) the amount of energy deposited. In this case the tomographic method can resolve in addition the presence of the agent or simply offer an image of increased contrast. The presence of agents can be detected as difference images, for example a difference image over baseline or by modifying the frequency or wavelength of the excitation energy; especially when agents of varying spectral absorption are utilized. These agents could be molecules, nano-particles and overall natural or synthetic constructs with preferred distribution characteristics, for example for distributing only in the vascular system, or for monitoring perfusion, permeability retention and other physiological functions. Additionally, they can have targeting capabilities, for example by targeting certain cells or certain tissue and cellular processes.

The inventors have found that the methodology and technique for the tomographic approach towards frequency domain optoacoustic and thermo-acoustic tomography can go beyond the limits of conventional FD-optoacoustic-imaging and fundamentally improve image quality by offering cross sectional tomographic images using measurements over different angular projections. Contrary to conventional TD based tomography, the invention is based on excitation with a continuously operating source device. Thus, the application of complex pulse sources is no longer required. Contrary to the complex and expensive laser sources typically employed in time domain, the frequency domain methodology attains the overall potential to operate with cost efficient and technically simpler light sources. Simultaneously, the excitation with high peak intensities is avoided, so that the thermo-/opto-acoustic imaging can be applied with sensitive objects, like in particular sensitive biological tissue or energy sensitive marker substances, for example light sensitive marker substances such as fluorochromes.

Furthermore, the FD thermo-acoustic tomographic imaging can lead to implementations of fast data acquisition and fast imaging capacity. In TD thermo-acoustics, the propagation of the electromagnetically induced acoustic pressure waves is determined by time of flight measurements, between the detectors employed and the structures imaged. The interval between two subsequent pulses illuminating the ROI has to be longer than the travel time of acoustic pressure waves of all absorbers within the ROI to ensure correct spatial resolution of absorbers. On the contrary, in the inventive FD thermo-acoustic tomography, acoustic pressure waves are continuously induced and collected, leading to up to 100% duty cycles, by dropping the direct time-space relationship which is based on distinct trigger events. The time-space relationship can be instead recovered through signal processing, such as correlating the modulation signal with the measured opto- and/or thermoacoustic response, simultaneously allowing an increased imaging velocity. Compared to TD or FD scanning systems, the present invention can further accelerate imaging by employing arrays of detectors, detecting in parallel signals travelling at multiple angular projections.

Furthermore, compared to previous FD opto-acoustic imaging implementations, based on two-dimensional (x-y) detector raster scans for image formation, the present invention teaches on methods to perform tomographic imaging using angular projections and mathematical inversion methods. In particular, FD optoacoustics was limited to x-y scan applications, resulting in depth-profilometric images (see S. Telenkov et al., cited above) with limited view scans. On the contrary, the invention provides tomographic imaging. Advantages connected to the tomographic approach over scanning geometries are signal to noise enhancement with regard to the adding of signals in a multiple projection scenario, improved sensitivity, image quality and quantification accuracy. In particular, the tomographic approach overcomes restrictions in scanning methods and makes signal acquisition and processing possible over multiple angular projections. Angular projections herein generally correspond to data collection over positions that are spread on the boundary of the object and provide signals that contain responses from partially overlapping targets in the object imaged. In contrast to scanning methods that form images by mechanically decomposing contributions from the target imaged (using the scan mechanism) the invention herein teaches on how to employ overlapping information in the frequency domain in order to formulate images, typically of higher accuracy and resolution compared to the ones produced by x-y scans. This is achieved by the utilization of mathematical methods that offer image reconstruction in the sense of mathematical inversion. In this case acoustic responses following e. g. RF/microwave/optical/magnetic excitation are collected over multiple projections around the object, and mathematically combined in a mathematical inversion scheme in order to yield images. Other advantages of tomographic FD thermoacoustic over lateral scanning include improved signal to noise enhancement resulting in better imaging contrast as compared to the single projection topology (x-y scans). Resolution also increases in the tomographic measurement scenario since target signals are detected at different views. Finally, resolution asymmetries offered by the limited view approach corresponding to x-y/lateral scans is overcome.

Advantageously, the differentiation of excitation energy (in particular RF/microwave/optical/magnetic) can lead to images of different contrast and application value. Optical energies can for example resolve hemoglobin, melanin, cells, optical (absorption) structures, functional characteristics of the tissue or object imaged. In addition photo-absorbing agents can be imaged, for example dyes, fluorochromes, nano-particles and molecules linked (conjugated) to photo-absorbing moieties. Correspondingly, RF or microwave imaging can reveal changes in water concentration, tissue electrical properties and RF/microwave absorbing moieties whereas magnetic excitation can be also employed to reveal magnetic properties of tissues, such as ones associated with iron modulation or metal-based agents and nanoparticles.

Generally, the imaged object includes at least one of biological tissue, biomedical material and industrial material. Depending on the type of excitation of mechanical waves in the ROI, the object can include a distribution of marker substances providing a distribution of mechanical wave sources. The marker substances comprise e. g. biomarker substances (molecules or particles with specific binding to biological tissue or parts thereof, with specific absorbance in optical or radiofrequency wavelength range), optical absorbers (absorbing molecules, proteins or particles with specific absorbance in the optical wavelength range) and/or radiofrequency absorbers (absorbing molecules or particles with specific absorbance in radiofrequency wavelength range).

Advantageously, multiple types of input modulation are available for varying the electromagnetic energy input during the continuous emission thereof. According to preferred variants of the invention, the input modulation includes at least one of frequency modulation, in particular chirp modulation, amplitude modulation, phase modulation and digital modulation (random modulation). These modulations have advantages for an efficient thermoacoustic wave generation and recovery of space information. The time dependency of the electromagnetic energy input can be selected in dependency on the practical conditions of the imaging apparatus. Thus, the input modulation may include at least one of a linear, logarithmic, sin-like, square-like, and triangle-like frequency modulation. A particularly preferred modulation type is the linear frequency modulation, such as a sine like modulation with linear increasing frequency. As an advantage, experiments of the inventors proved highest SNR with the sine-like linear frequency modulation.

With preferred applications of the invention, modulation frequencies can be chosen in dependency on the size (or: frequency response) of the structures in the ROI and the bandwidth of the acoustic detector elements. In other words, imaging of smaller structures is preferably done with a modulation waveform having higher frequencies than imaging of larger structures. This represents furthermore an advantage of the frequency domain photoacoustic tomography approach because the frequencies can be adjusted to the detection bandwidth and size of the objects in the ROI.

For detecting the mechanical wave response signals in the ROI along multiple projection directions, the detector device preferably includes at least one acoustic detector element which is sensitive to mechanical stress and which is movable relative to the ROI. The acoustic detector element is a transducer element as known in prior art of ultrasound or photo/thermoacoustic imaging. Moving at least one acoustic detector element has advantages in terms of selecting the number and/or orientation projection directions. Alternatively or additionally, the detector device may include a detector array with multiple acoustic detector elements. The acoustic detector elements comprise transducer element which are fixedly distributed around the ROI. This embodiment has advantages in terms of simultaneous collection of mechanical wave response signals. Furthermore, an optical or interferometric device can be used for collecting the mechanical wave response signals.

In particular, the acoustic pressure waves are referred to as mechanical waves induced in the ROI following electromagnetic or optical stimulation and can be detected by at least one acoustic element. For FD based optoacoustic tomography with optical excitation, the detector device preferably is comprised of at least one detection element made of PZT and/or PVDF. With magnetic/RF/microwave excitation, the detector device preferably contains at least one mechanical pressure detection element which is advantageously based on an optical interferometric detector with respect to the electromagnetic interferences originating from an RF emitting source device. Advantageously, acquisition of mechanical waves with an optical interferometric detector decouples the electromagnetic excitation from the optical detection, thus minimizing distortions coming from electromagnetic interferences on the detection path and increases the bandwidth of the acquired acoustic signal, thus improving reconstruction performance.

According to a further preferred embodiment of the invention, the source device includes an array of continuously emitting sources. The source device is adapted for emitting the electromagnetic energy input along multiple irradiation directions into the ROI. Advantageously, a homogeneous generation of mechanical waves in the ROI is obtained. With an alternative embodiment of the invention the emitting sources of the array can be adapted for emitting the electromagnetic energy input with different wavelengths. The application of multiple sources favors a frequency, time or phase coding of each source in order to perform simultaneous (distinguishable) excitation of targets.

Advantageously, the invention can be implemented with both of optoacoustic imaging and other thermo-acoustic imaging. Thus, according to a particularly preferred embodiment of the invention (optoacoustic or OA embodiment), the source device is adapted for continuously emitting the electromagnetic energy input in an optical wavelength range including at least one of UV, VIS and IR wavelength ranges. With this preferred example, the ROI is illuminated with an intensity modulated CW light source and induced mechanical pressure waves following optical absorption are acquired in an imaging plane with the detector device. Signals representing the detected acoustic pressure waves originating from optical sources within the region of interest are processed to tomographic image data, representing the distribution of optical absorbers in the imaging plane. Contrary to other frequency domain optoacoustic imaging modalities, data is collected over multiple projections around the object, resulting in the tomographic data set. Therefore, cross sectional tomographic images can be reconstructed from the detected acoustic pressure waves.

With preferred variants of the OA embodiment, the source device comprises at least one of an amplitude modulated CW laser and an amplitude modulated light emitting diode, or a similar light source. Alternatively or additionally, the source device is provided with at least one of an acousto-optic modulator, electro-optic modulator, a mechanical chopper, and an electrically modulated power source.

According to a particularly advantageous embodiment of the invention, the excitation source is a laser diode with optical emission in the NIR/IR region of the electromagnetic spectrum. The optical excitation of the target is achieved e. g. by a continuous wave (CW) light source with emission wavelength in the optical band and near-infrared band. Generally, the excitation wavelength of the frequency domain optoacoustic scanner can also be performed with lower wavelength or higher wavelength, but with respect to the absorption spectrum of water and intrinsic optical contrast of biological tissue which is mainly attributed to hemoglobin in oxygenated and deoxygenated states, preferably wavelengths in the near-infrared and infrared region are utilized for excitation.

According to an alternative particularly preferred embodiment of the invention (thermoacoustic or TA embodiment), the source device is adapted for continuously emitting the electromagnetic energy input in a radiofrequency range, in particular microwave or radiofrequency range, or THz radiation range. With this preferred example, the ROI is illuminated with a focused RF−, magnetic and/or microwave field and subsequently induced mechanical pressure waves due to absorption of locally dissipated electromagnetic energy are detected with the detector device sensitive to mechanical stress and oscillations. Signals representing the detected acoustic pressure waves originating from RF/magnetic/microwave absorbers within the ROI are processed to image data, representing the distribution of RF/magnetic/microwave absorbers in the imaging plane. Imaging is facilitated over multiple projections around the object, resulting in the tomographic data set of the target. Therefore, cross sectional tomographic images can be reconstructed from the detected acoustic pressure waves.

Advantageously, the TA embodiment allows for deep tissue penetration in tissues since e. g. biological matter has low absorption coefficients in the low MHz region and is furthermore almost transparent to magnetic fields. Advantageously, the exogeneous administration of contrast enhancing agents and probes featuring RF/magnetic/microwave absorption can introduce defined absorption markers in the target tissue.

With preferred variants of the TA embodiment, the source device comprises at least one radiofrequency source emitting in the low MHz region, in particular in the 0.1 MHz to 100 MHz region of the electromagnetic spectrum. However, the excitation frequency is not limited to the mentioned frequency band but can also be extended to lower and higher frequencies. Additionally or alternatively, the source device comprises at least one energy coupling element, e.g. a magnetic coil device and/or an antenna device.

Preferably, the source device is a device which facilitates CW RF/magnetic excitation with either dominant electric or magnetic field contributions, adapted for the FD thermoacoustic tomography. As an example, magnetic excitation of target tissue is achieved by a device which in particular is adapted for generating dominating magnetic fields. The device is designed for narrowband excitation of magnetic nanoparticles (ferro, ferri, para, dia, and/or superparamagnetic type) which exhibit localized absorption of (electro)magnetic energy in the above low MHz region.

According to preferred embodiments of the invention, the imaging method can include a step of introducing a distribution of marker substances into the object. The marker substances may comprise fluorescent proteins, chromophoric or fluorescent molecules, particles (nano-, micro-), photodynamic therapy agents, paramagnetic particles, super-paramagnetic particles, ferromagnetic particles, diamagnetic particles, magnetic loss particles, carbon particles, ceramic particles, electrically conducting particles, particles from noble metals, semiconducting particles and/or activatable substrates.

With the preferred TA embodiment, application of extrinsically administered contrast agents and probes featuring significant RF/magnetic/microwave absorption is of particular importance for enhancing contrast and increasing information content. In particular for excitation with dominating magnetic fields where absorption in biological tissue is negligible, contrast enhancers add essential information to the imaging data. Candidates for exogenously injected agents into a specific region of the tissue are particles which feature significant RF energy absorption such as magnetic nanoparticles (U.S. Pat. No. 4,770,183,) which were already employed in MRI studies as well as therapeutic agents used for hyperthermia (thermal ablation) applications (U.S. Pat. No. 7,510,555 B2). Besides, agents and probes which are characterized by radiofrequency or magnetic absorption may be utilized with the developed method. Among these agents can also be molecules, micelles or loaded cells or any kind of particle and substance which exhibits electromagnetic absorption. Functionalized particles which are conjugated to antibodies can also be used as agents for targeted local stimulation of tissue.

The imaging apparatus can also be implemented as a hybrid FD optothermoacoustic tomography scanner, combining optical excitation and RF/magnetic/microwave excitation in one system. Advantageously, the system combines intrinsic optical and RF/magnetic/microwave contrast in one image and is also capable of resolving optical and RF/magnetic/microwave contrast agents.

According to a further embodiment of the invention, the imaging method can include a step of operating the source device in a treatment mode with an increased level of electromagnetic energy input and subjecting the object to thermal treatment by the increased level electromagnetic energy input.

According to yet another embodiment of the invention, at least one of the detector device and the source device or parts thereof are configured to be inserted inside a blood vessel or another hollow organ for intra-cavity imaging, for example intravascular imaging, colonoscopic imaging, gastro-intestinal track imaging, transurethral imaging etc. In other words, according to a first variant, at least one detector element or detector array of the detector device and at least one emitter or an emitter array of the source device are configured to be inserted inside the hollow organ for tomographic imaging. According to a second variant, at least one detector element or detector array of the detector device is configured to be arranged within the hollow organ, while the source device or parts thereof is configured to be operated from the outside of the hollow organ. According to a third variant, at least one emitter or an emitter array of the source device is configured to be arranged within the hollow organ, while the detector device is configured to be operated from the outside. Preferably, at least one of the detector device and the source device or parts thereof, i. e. at least one detector element or detector array of the detector device and at least one emitter or an emitter array of the source device, are arranged in a hand held unit for tomographic data acquisition.

Further generally preferred features of the imaging apparatus of the invention include a reconstruction unit processing the data and reconstructing a tomographic image of a distribution of electromagnetic energy absorbers within the ROI, and/or a carrier device being arranged for accommodating the object. The carrier device is configured for moving the object relative to the detector device. Furthermore, parts of the source device and object to be imaged can be electromagnetically isolated and decoupled from the detector device, reducing and preferably avoiding electromagnetic interference on the signal and measurement path. Furthermore, a matching medium can be provided for better RF/magnetic/microwave coupling between the source device and the object and simultaneously for increased coupling between the object and the detector device. The matching medium can be comprised of a fluid, gel or oil.

While tissue imaging correspond herein to a preferred application, the invention can be applied to a much wider area of imaging and sensing including tomographic imaging of foods, fluids and environmental applications. The invention can lead to non-invasive tomographic imaging and biochemical or structural sensing of fruits, foods, milk and other fluids, in in-vitro sensing applications of biological specimen for example in examining the quality of blood or industry fluids employed in machinery operation. Similarly plant imaging and environmental material including water, soil and compressed atmospheric or car emission gases can be similarly analyzed.

Finally of fundamental importance in the invention taught is the use of mathematical methods for image formation, in the context of mathematical image inversion. Mathematical inversion refers to allocated signals collected at multiple positions (projections) along lines or volumes (projections) inside tissue. By overlapping a number of measured signals an image can then be formed. This step is not present in conventional x-y raster scan methods but can significantly contribute to improvements in image performance. This can be achieved by employing back-projection methods or more notably when the inversion employs models that describe the physical phenomena associated with wave propagation (thermal, acoustic and photon/light) in tissue and the associated energy absorption and wave detection processes. Model based methods can further incorporate features of the hardware employed in image formation further improving the accuracy.

### BRIEF DESCRIPTION OF THE DRAWINGS

Further details and advantages of the invention are described in the following with reference to the attached drawings, which show in:

FIGS. 1 to 5: schematic illustrations of embodiment of an imaging apparatus according to the invention; and

FIGS. 6 to 7: graphic representations of experimental results obtained with the invention.

### PREFERRED EMBODIMENTS OF THE INVENTION

Preferred features of the imaging apparatus and method for thermo-acoustic tomographic imaging according to the invention are described in the following with particular reference to the mathematical basics of the thermo-acoustic signal generation and image data reconstruction (1.), embodiments of the imaging apparatus (2.), preferred applications (3.) and experimental results (4.). Exemplary reference is made to the imaging of biological tissue. It is emphasized that the implementation of the invention is not restricted to the illustrated examples but rather possible in particular with modified arrangements of the imaging apparatus, further applications and other objects, like e. g. workpieces. In particular, the illustrated arrangements can be replaced by modified designs, wherein at least one detector element or detector array of the detector device and at least one emitter or an emitter array of the source device are arranged in a hand held unit.

### 1. Thermo-Acoustic Signal Generation and Image Data Reconstruction

The governing equations for thermo-acoustic wave generation and propagation can be expressed as

$$\begin{matrix}\frac{1}{α} \frac{\partial }{\partial t} T ( \vec{r} , t ) - {∇}^{2} T ( \vec{r} , t ) = \frac{1}{K} H ( \vec{r} , t ) & ( 1 ) \\ ( {∇}^{2} - \frac{1}{{v}_{s}^{2}} \frac{{\partial }^{2}}{\partial {t}^{2}} ) p ( \vec{r} , t ) = - \frac{β}{κ {v}_{s}^{2}} \frac{{\partial }^{2}}{\partial {t}^{2}} T ( \vec{r} , t ) & ( 2 )\end{matrix}$$

with the Temperature T at position r̅ and time t, the thermal diffusivity

$$α = \frac{K}{ρ {C}_{v}}$$

with the thermal conductivity K, the isothermal compressibility

$$κ = \frac{{C}_{p}}{ρ {v}_{s}^{2} {C}_{v}} ,$$

the mass density ρ, the specific heat capacities Cᵥ and Cₚ at constant volume and pressure, respectively, the pressure p induced at position r̅ and time t, the heating function H, the volume coefficient of thermal expansion β and the speed of sound vₛ. Substituting equation (1) in (2) without neglecting spatial heat diffusion yields

$$( {∇}^{2} - \frac{1}{{v}_{s}^{2}} \frac{{\partial }^{2}}{\partial {t}^{2}} ) p ( \vec{r} , t ) = - \frac{β}{{C}_{p}} \frac{\partial }{\partial t} ( K {∇}^{2} T ( \vec{r} , t ) + H ( \vec{r} , t ) \qquad ( 3 )$$

and in frequency domain (transition from time domain

$$p {\to }^{FT} \hat{p}$$

into frequency domain with the Fourier transform FT)

$$( {∇}^{2} + \frac{{ω}^{2}}{{v}_{s}^{2}} ) \hat{p} ( \vec{r} , ω ) = - j ω \frac{β}{{C}_{p}} ( K {∇}^{2} \hat{T} ( \vec{r} , ω ) + \hat{H} ( \vec{r} , ω ) ) . \qquad ( 4 )$$

Equation (4) can be rewritten as

$$( {∇}^{2} + {k}^{2} ) \hat{p} ( \vec{r} , ω ) = - j ω \frac{β}{{C}_{p}} ( \hat{u} ( \vec{r} , ω ) ) \qquad ( 5 )$$

with the source function

\hat{u}(\vec{r},ω)=K∇²\hat{T}(\vec{r},ω)+H(\vec{r},ω) (6)

and the wave number

$$k = \frac{ω}{{v}_{s}} .$$

Using the Green-function approach, the solution to the Helmholtz equation (5) can be expressed as

$$\hat{p} ( \vec{r} , ω ) = - \frac{j ω β}{4 π {C}_{p}} {\int }_{V} \frac{1}{\vert \vec{r} - {\vec{r}}^{′} \vert} \hat{u} ( {\vec{r}}^{′} , ω ) exp ( j k \vert \vec{r} - {\vec{r}}^{′} \vert ) {d}^{3} {\vec{r}}^{′} \qquad ( 7 )$$

### Heating Function for Optical Excitation

Depending on the type of excitation, the heating function for the optical case in the frequency domain can be expressed as

Ĥ(\vec{r},ω)=∂μI₀\hat{F}(ω)exp(−μ|\vec{r}−\vec{r}₀|) (8)

with the optical absorption coefficient μ, the dimensionless energy conversion efficiency ∂, the modulation waveform in the Fourier domain \hat{F}(ω) and the irradiance I₀.

### Heating Function for RF/Microwave Excitation

Regarding RF/microwave excitation, the heating function can be written as

\hat{H}(\vec{r},ω)=∂\hat{P}(\vec{r},ω)exp(−α_{EM}|\vec{r}−\vec{r}₀|) (9)

with the power dissipation

$$\hat{P} ( \vec{r} , ω ) = \frac{σ ( \vec{r} )}{2} {\vert \hat{\vec{E}} ( \vec{r} , ω ) \vert}^{2} + π f {ɛ}_{0} {ɛ}_{r}^{″} ( \vec{r} ) {\vert \hat{\vec{E}} ( \vec{r} , ω ) \vert}^{2} + π f {μ}_{0} {μ}_{r}^{″} ( \vec{r} ) {\vert {\hat{\vec{H}}}_{mag} ( \vec{r} , ω ) \vert}^{2} \qquad ( 10 )$$

and the RF attenuation coefficient

$${α}_{EM} = ω \sqrt{( \frac{μ ɛ}{2} \sqrt{1 + {( \frac{σ}{ω ɛ} )}^{2}} - 1 )} , \qquad ( 11 )$$

with the electric field strength \vec{Ê}(\vec{r},ω), the magnetic field strength \vec{Ĥ}_{mag}(\vec{r},ω), electric conductivity σ(\vec{r}), the permittivity of free space ε₀, the permeability of free space μ₀, the complex relative electric permittivity εᵣ=εᵣ′−jεᵣ″, the complex relative magnetic permeability μᵣ=μᵣ′−jμᵣ″, the imaginary part of the complex relative electric permittivity εᵣ″ describing electromagnetic losses resulting from vibrational motion of atoms or molecule dipoles and the imaginary part of the complex relative magnetic permeability μᵣ″ relating to electromagnetic losses from magnetic polarization from magnetic moments.

### Tracing Back Spatial Information/Waveform Modulation/Frequency Domain Processing

Preferably, the spatial distance of absorbers within the ROI is traced back with algorithms known from pulse compression schemes in Radar technique (M. Skolnik, Radar Handbook, Third Edition, McGraw Hill, 2008/M. Skolnik, Introduction to Radar Systems, Third Edition, McGraw Hill, 2001). In pulse compression mode, long coded pulses are transmitted and correlated to the received signal. Applying pulse compression to the frequency domain thermo-acoustic methodology, targets are continuously illuminated with modulated waveforms at durations in the order of preferably t≈ms, in general t≧10 μs, but the modulated waveform width can also be t≈1 s and longer. The modulation type is preferably linear but can also be logarithmic, sin-like, square-like, triangle-like or be modulated with another similar waveform. An example of a linear frequency modulated waveform is given by equation

x(t)=A cos [2πf₀t+πbt²] (12)

with the initial frequency f₀, the amplitude A and the sweep rate b. Correlating the known excitation waveform type with the measured thermo-acoustic mechanical wave response signals yields the phase and accordingly the time delay of absorbers within the detection range of the acoustic detection element.

Correlation of excitation waveform l(\vec{r},t) with the detected thermo-acoustic response p(\vec{r},t) can be advantageously performed in frequency domain since the cross correlation yields a product operation in the frequency domain

Time domain:

$${p}_{c} ( \vec{r} , t ) = {\int }_{- ∞}^{∞} {l}^{*} ( \vec{r} , τ ) \cdot p ( \vec{r} , t + τ ) d τ \qquad ( 13 )$$

Frequency domain: \hat{p}_{c}(\vec{r},ω)=\hat{l}*(\vec{r},ω)·\hat{p}(r,ω). (14)

Furthermore, signal processing like filtering can be advantageously performed in the frequency domain with respect to time and memory efficient imaging performance. Advantageously, the pulses are modulated with frequencies matching the detection bandwidth of the transducers. For narrowband acoustic detection elements like PZT based transducers, the excitation frequency band is chosen according to the frequency sensitive band of the detectors whereas for broadband acoustic detectors the excitation frequencies are advantageously matched to the broadband response.

### Image Reconstruction

Cross sectional thermo-acoustic images within a tomographic measurement topology can be reconstructed with the frequency domain equation (7)

$$\hat{{p}_{c}} ( \vec{r} , ω ) = - \frac{j ω β}{4 π {C}_{p}} {\int }_{V} \frac{1}{\vert \vec{r} - {\vec{r}}^{′} \vert} {\hat{u}}_{c} ( {\vec{r}}^{′} , ω ) exp ( j k \vert \vec{r} - {\vec{r}}^{′} \vert ) {d}^{3} {\vec{r}}^{′} \qquad ( 15 )$$

with the source function û_{c}(\vec{r},ω) and the measured acoustic signal \hat{p}_{c} (\vec{r},ω) after cross correlation. Basically, equation (15) has to be solved for the unknown source function, e.g. by inversion of the equation. There are different approaches to solve for the source function, however in what follows four reconstruction methods are presented of which three are operating in frequency domain and the first method performs the inversion for the source function in time domain using the frequency domain cross correlated signals from equation (14).

### Modified Backprojection

A first method to reconstruct tomographic images from cross correlated signals is the modified backprojection formula as given by (M. Xu and L. H. Wang, Universal back-projection algorithm for photoacoustic-computed tomography, Phys. Rev., vol. E71, no. 1, pt. 2, p. 016706 (2005))

$${u}_{c} ( \vec{r} , t ) = - \frac{1}{2 π {c}^{2} Γ} {\int }_{S}^{ } t [ \frac{\partial {p}_{c} ( {\vec{r}}_{0} , t = \vert \vec{r} - {\vec{r}}_{0}^{′} \vert {c}^{- 1} )}{\partial t} - \frac{{p}_{c} ( {\vec{r}}_{0} , t = \vert \vec{r} - {\vec{r}}_{0}^{′} \vert {c}^{- 1} )}{t} ] d S \qquad ( 16 )$$

with the Grüneisen parameter Γ=βvₛ²Cₚ⁻¹. This equation is basically a (2D) inversion formula for the time domain analytical solution to the wave equation (3) given by

$$p ( \vec{r} , t ) = \frac{ β}{4 π {C}_{p}} \frac{\partial }{\partial t} \int \frac{u ( \vec{r} , t )}{\vert \vec{r} - {\vec{r}}^{′} \vert} d A . \qquad ( 17 )$$

In order to form a cross sectional image, the detected thermoacoustic mechanical wave response signals are transformed into the frequency domain where preprocessing steps like e.g. filtering and cross correlation according to equation (14) are implemented. Since the cross correlation yields the phase shift between two signals, the FD cross correlation \hat{p}_{c}(\hat{r},ω) is transformed back into the time domain where the phase shift is converted into a time shift, representing the distance of absorbers in the imaging plane from the detector. Finally, the time domain cross correlated signal p_{c}(\vec{r},t) is used to feed the modified back-projection algorithm in equation (16).

### Model-Based Reconstruction

Another solution is a model based approach similar to the algorithm proposed in (Amir Rosenthal, Daniel Razansky, and Vasilis Ntziachristos, Fast Semi-Analytical Model-Based Acoustic Inversion for Quantitative Optoacoustic Tomography, IEEE Transactions on Medical Imaging 29(6), 1275-1285 (2010)). Unlike the preceding method, this reconstruction algorithm operates in frequency domain, thus giving a solution to equation (15) which is based on a matrix equation that can be inverted for the unknown source function. This approach specifies a grid map upon which the thermo-acoustic image is projected. The model based equation is defined as

\hat{p}_{c}=Mŝ (18)

with the vector \hat{p}_{c} containing the measured thermo-acoustic signals after cross correlation, the forward model matrix M describing the imaging system, detection geometry plus thermo-acoustic signal generation and propagation. The integral in equation (15) can be interpolated analytically over arcs, building the model matrix M which is to be inverted. The vector ŝ contains predefined image pixels and is the unknown which is to be solved for. Various methods for the mathematical problem of matrix inversion exist; a standard inversion type is based on minimizing the mean square error

ŝ=arg min∥\hat{p}_{c}−Mŝ∥² (19)

with e.g. the LSQR algorithm or the Moore-Penrose pseudo-inverse. Transforming ŝ back into the time domain results in the thermo-acoustic image which represents the distribution of electromagnetic (e.g. microwave/RF/magnetic/optical) absorbers in the imaging plane. An advantage of the model based approach is that the imaging geometry and acoustic propagation (e.g. frequency dependent acoustic attenuation) and detection (e.g. frequency response of detectors) can be modeled thus creating accurate image reconstructions.

The model based approach can also be implemented in the time domain using the TD equivalent

$${\hat{p}}_{c} {}^{{FT}^{- 1}} {p}_{c}$$

of the cross correlated signal. Instead of the FD equation (15), the time domain model based approach solves for equation (17), thus building up the geometric, acoustic and detection model in the time domain. The model matrix is then given by

p_{c}=Ms (20)

with a similar inversion scheme

s=arg min∥p_{c}−Ms∥². (21)

Similarly, the frequency domain advantages like accurate image reconstructions are also valid for the time domain approach.

### Frequency Domain Thermoacoustic Tomographic Reconstruction Using Fourier Diffraction Theorem

Yet referring to another reconstruction algorithm, cross sectional images with the invention can advantageously be reconstructed using Diffraction Tomography methods (as proposed in Kak and Slaney). This approach is based on wave solutions to diffractive sources and employs the Fourier Diffraction theorem as described by Baddour in 2008 (Natalie Baddour, Theory and analysis of frequency-domain photoacoustic tomography, J. Acoust. Soc. Am. 123 (5), 2577-2590 (2008)). It assumes that an object o(x, y) in the x-y imaging plane which is illuminated with a plane wave results in a scattered field of which the spatial 2D Fourier transform yields the object ô(ωₓ,ω_{y}) in the Fourier domain. The scattered field is measured along a line either in transmission or reflection mode and the resulting Fourier domain signal is a half circular arc for both reflection and transmission. The overall potential as compared to other frequency domain optoacoustic imaging methods which operate in a x-y lateral scanning geometry is that the Diffraction Tomography methodology takes advantage of multiple angular projection data in order to fill up the frequency domain. In other words, the tomographic detection over different projections (at least 60° coverage of a full circle) result in semicircular arcs in the Fourier (ωₓ,ω_{y}) plane, thus filling the frequency domain with arcs corresponding to the respective Fourier transformed projection of the detected signals. Furthermore, another advantage associated with the Diffraction Tomographic reconstruction is that the light propagation, the acoustic propagation as well as the thermal propagation in the object can be modeled, thus combining the Diffraction Tomography reconstruction with the model based approach. Thus, geometrical (e.g. detection and illumination geometry, including frequency response of detectors) and operational (e.g. acoustic, thermal and light propagation) parameters can be integrated in a forward model matrix which is used for image reconstruction.

### Fourier Domain Reconstruction Using Hankel Transformation

Finally, another method employs the Hankel transform to solve the inversion in equation (15) in the frequency domain. Considering a cylindrical geometry with a polar coordinate system (see Yuan Xu, Minghua Xu, and Lihong V. Wang, Exact Frequency-Domain Reconstruction for Thermoacoustic Tomography—II: Cylindrical Geometry, IEEE Transactions on Medical Imaging 21(7), 829-833 (2002)), equation (15) can be expressed as

$$\hat{{p}_{c}} ( \vec{r} , k ) = - \frac{k βsgn ( k )}{8 π {C}_{p}} {\int }_{V} {\hat{u}}_{c} ( {\vec{r}}^{′} , k ) {d}^{3} {\vec{r}}^{′} \cdot {\int }_{- ∞}^{∞} exp [ - j {k}_{z} ( {z}^{′} - z ) ] d {k}_{z} \cdot {\sum }_{m = - ∞}^{m = ∞} A ( m , μ {ρ}^{′} , μ ρ ) exp [ - Im ( {ϕ}^{′} - ϕ ) ] \qquad ( 22 )$$

employing the relationship

$$\frac{exp ( - j k \vert \vec{r} - {\vec{r}}^{′} \vert )}{4 π \vert \vec{r} - {\vec{r}}^{′} \vert} = \frac{- j}{8 π} {\int }_{- ∞}^{∞} exp [ - j {k}_{z} ( {z}^{′} - z ) ] d {k}_{z} \cdot {\sum }_{m = - ∞}^{m = ∞} A ( m , μ {ρ}^{′} , μ ρ ) exp [ - Im ( {ϕ}^{′} - ϕ ) ] \qquad ( 23 )$$

where the function A is defined as

A(m,μρ′,μρ)=Jₘ(μρ′)Hₘ²(μρ) (24)

with the mth-order Bessel function Jₘ and the second-kind Hankel function Hₘ². Performing the Fourier transforms with respect to the polar coordinates φ and z yields equation

$$\hat{{p}_{c}} ( m , {k}_{z} , k ) = \frac{k β {H}_{m}^{2} ( μ ρ )}{8 π {C}_{p}} {\int }_{0}^{∞} {\hat{u}}_{c} ( m , {k}_{z} , {ρ}^{′} ) {J}_{m} ( μ {ρ}^{′} ) {ρ}^{′} d {ρ}^{′} , \qquad ( 25 )$$

where û_{c}(m,k_{z},ρ′) corresponds to the φ and z dependent fourier transform of û_{c}(\vec{r}′,k), and accordingly \hat{p}_{c}(m,k_{z},k) relates to the φ and z dependent fourier transform of \hat{p}_{c}(\vec{r}′,k). Equation (25) can be solved for the source function û_{c}(m,k_{z},ρ′) with the inverse Hankel transformation (see Yuan Xu, Minghua Xu, and Lihong V. Wang, Exact Frequency-Domain Reconstruction for Thermoacoustic Tomography—II: Cylindrical Geometry, IEEE Transactions on Medical Imaging 21(7), 829-833 (2002)), thus yielding

$$\begin{matrix}\hat{{u}_{c}} ( m , {k}_{z} , k ) = \frac{8 π {C}_{p}}{β} {\int }_{0}^{∞} \frac{μ {\hat{p}}_{c} ( m , {k}_{z} , k ) {J}_{m} ( μ {ρ}^{′} )}{{kH}_{m}^{2} ( μ ρ )} d μ or & ( 26 ) \\ \hat{{u}_{c}} ( m , {k}_{z} , k ) = \frac{8 π {C}_{p}}{β} {\int }_{{k}_{z}}^{∞} \frac{{\hat{p}}_{q} ( m , {k}_{z} , k ) {J}_{m} ( μ {ρ}^{′} )}{{H}_{m}^{2} ( μ ρ )} d k & ( 27 )\end{matrix}$$

with the relationship μ=sign(k)\sqrt{|k²−k_{z}²|}. The inverse Fourier transform to û_{c}(m,k_{z},k) with respect to m and k_{z} finally results in the source function which is used to form a thermoacoustic cross sectional image of RF/microwave/magnetic/optical absorbers in the imaging plane.

### 2. Embodiments of the Imaging Apparatus and Method

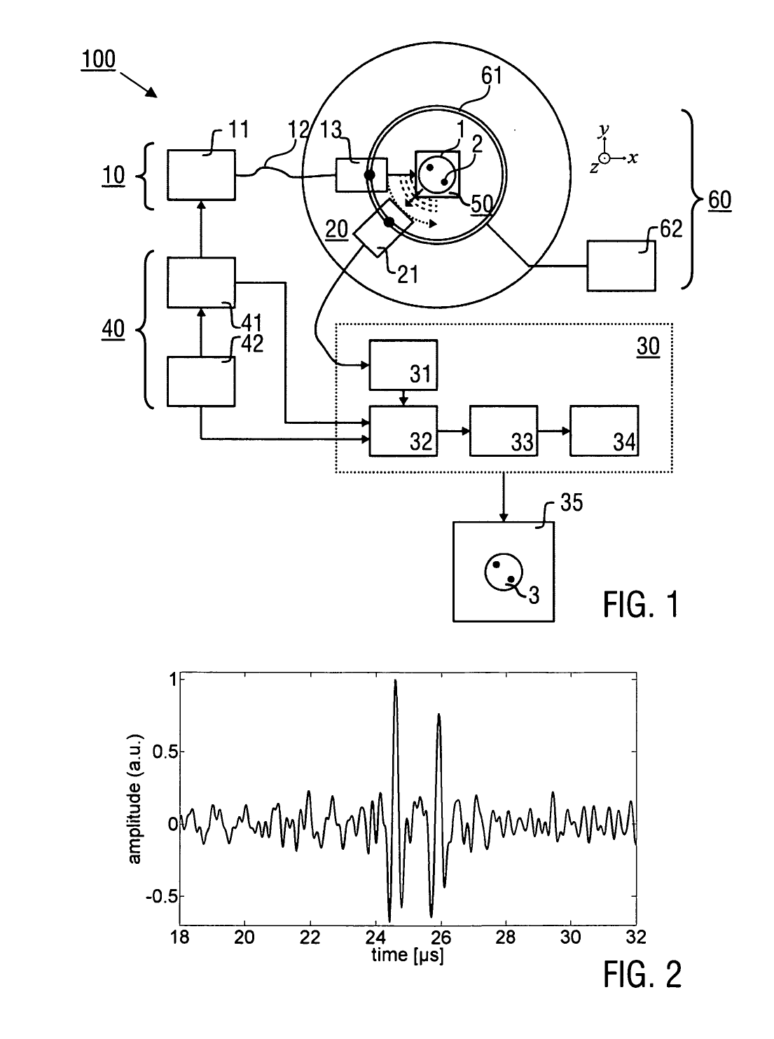

FIG. 1 shows a schematic drawing of a preferred embodiment of a frequency domain optoacoustic tomographic imaging apparatus 100 including a source device 10, a detector device 20, an image data acquisition and processing device 30, a control device 40, a carrier device 50 accommodating an object 1 with ROI 2, and a motion device 60.

The source device 10 comprises a laser source 11 for illuminating the object 1. The laser source 11 has advantages in particular with respect to the preferred application of biomedical imaging. As an example, the laser source 11 comprises a temperature-stabilized CW laser (Omicron A350, Omicron-Laserage Laserprodukte, GmbH, Germany), which was used also for the experimental results outlined below. The laser source 11 emits an amplitude modulated CW beam at 808 nm providing the electromagnetic energy input directed to the object 1. Amplitude modulation is obtained with the control device 40 including a signal generator 41, which provides a control signal modulating the output intensity of the laser source 11 by controlling an electrically modulated power source thereof or an acousto-optic modulator, electro-optic modulator or a mechanical chopper (not shown). The output intensity is modulated with a modulation frequency above 0 Hz and up to the MHz range, e. g. up to 350 MHz. Light from the laser source 11 is guided onto the object 1 using a light guiding optical fiber 12. Alternatively or additionally, mirrors can be used for guiding the modulated CW beam. The light beam can furthermore be collimated with a lens 13 (collimation lens) to focus the modulated CW beam on the object 1.

The modulation frequencies can be chosen in dependency on the size of the structures in the ROI and the bandwidth of the acoustic detector elements along the following rule. A 10 MHz transducer with a bandwidth of 70%, i.e. an effective 6 dB detection bandwidth of 6.5 MHz to 13.5 MHz would be convenient for imaging structures as small as ˜100 μm. Therefore, the modulation signal could be a sine like linear frequency modulated signal, starting at 6.5 MHz going up to 13.5 MHz. On the other hand, imaging of bigger structures (˜300 μm) would result with lower frequencies like 1 to 5 MHz with a 3.5 MHz transducer whereas tiny structures in the range of 10 μm would require a 100 MHz transducer with modulation frequencies centered around 100 MHz.

It is to be noted that the illumination is not limited to the laser source 11 but advantageously is comprised of at least one element, ideally illuminating the object homogeneously. The source device 10 can be implemented as any kind of light source, e. g. a light emitting diode (LED) or an array of LED's can be used. Furthermore, the source device 10 is preferably employing multiple wavelengths for multispectral excitation of the object 1.

Acoustic pressure signals are detected with the detector device 20 which is sensitive to mechanical pressure waves induced in the object 1 by electromagnetic, in particular optical absorption. The detector device 20 is comprised of at least one detector element 21 but preferably of multiple elements, e.g. a phased detector array. Due to the continuous modulated illumination, the mechanical pressure waves represent CW thermo-acoustic signals, which are coupled from the object 1 to the detector element 21 via a coupling medium (not shown). For frequency domain optoacoustic imaging, the detector device 20 is ideally based on at least one PZT/PVDF transducer, but can also be an optical detector like an optical interferometric mechanical stress detector. The detector device 20 is chosen according to the size of the object 1 and the excitation frequency (modulation frequency) of the illumination.

The light paths from the lens 13 to the object 1 and from the object 1 to the detector element 21 span an imaging plane through ROI 2. Tomographic image data are collected along a plurality of projection directions in the imaging plane. The number and/or distribution of projection directions can be selected as it is known from conventional tomography techniques.

The image data acquisition and processing device 30 includes an amplifying unit 31 (optionally provided), which is used for a pre-amplification of the induced CW opto-acoustic signals, a data acquisition device 32, a signal processing and storing unit 33 and an image reconstruction unit 34 connected with an output device 35, providing the reconstructed image which represents an equivalent map of optical absorption within the ROI 2 of the object. The output device 35 can comprise at least one of a display screen, e. g. of a computer, a printer or a data storage device. The data acquisition device 32 is detecting the optoacoustic signals as induced by the laser source 11. To this end, the data acquisition device 32 is connected with the detector element 21. Furthermore, the data acquisition device 32 is connected with a trigger generator 42 of the control device 40. The trigger generator 42 provides a reference signal which is synchronized with the control signal of the signal generator 41 which is also connected to the data acquisition device 32. The raw optoacoustic signals are processed and stored by the signal processing and storage unit 33, which performs a correlation processing between the optoacoustic signal and the reference signal and optionally furthermore fulfills spectral filtering tasks. The image 3 of ROI 2 is reconstructed in the image reconstruction unit 34 and displayed on the output device 35.

The control device 40 includes the signal generator 41 and the trigger generator 42 operating as a synchronization device. The signal generator 41 provides the waveform modulation of the laser source 11 which can be of e. g. linear, logarithmic, sin, square, triangle characteristic. The laser source 11 and the data acquisition device 32 are both synchronized by the trigger generator 42 which launches the measurements.

The object 1 is arranged on the carrier device 50, which includes e. g. a platform and/or carrying rods for arranging the object 1 relative to the source device 10. A tomographic data set for cross sectional views of the object 1 is acquired along multiple projection directions through the object 1. In the embodiment of FIG. 1, the projection directions are set by simultaneously rotating the light guiding optical fiber 12 with the lens 13 and the detector element 21 around the object 1 with the motion device 60. The motion device 60 includes a rotation stage 61 which is controlled by a motion controller 62. Alternatively, tomographic data acquisition can also be realized with a rotation of the object 1 in the imaging plane. Furthermore, volumetric images of the object 1 can be generated by translating either the object 1 perpendicular to the imaging plane or moving at least the detector device 20 relative to the object 1 along an elevation axis perpendicular to the imaging plane.

FIG. 2 illustrates an example of a processed signal after cross correlating the modulation signal (reference signal) with the measured optoacoustic signal. The invention has been tested on phantoms with different size and material, e. g. two graphite rods placed 2 mm apart. The imaging was performed with the preliminary setup depicted in FIG. 1 with a linear frequency sweep modulation of the laser ranging from 1 MHz to 5 MHz (see equation cross correlation). The detector device 20 consisted of a single detector element 21 based on PZT with a central frequency of 3.5 MHz, matching the frequency sweep of the laser modulation. Optoacoustic data with the linear frequency sweep was detected around the graphite rods at 180 projections in 2° steps. The post processed signal in FIG. 2, which is one projection out of 180 projections around the object, resulting in a total 360° full circle view of the target, shows the radial distance from each absorber from the detector element 21 with a characteristic sinc shape, which is the mathematical solution to the cross correlation of two frequency modulated chirps. The corresponding image reconstruction after signal processing, which is performed in frequency domain by converting the measured time domain optoacoustic signal and the modulation signal into frequency domain according to equation (14) revealed a cross sectional, tomographic view of the two graphite rods.

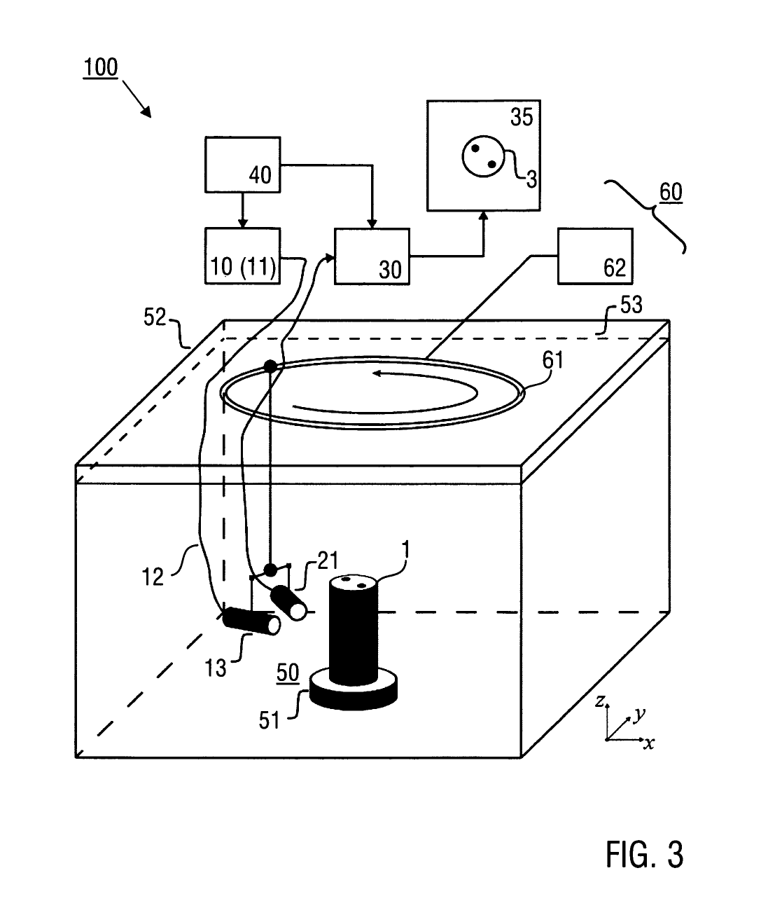

FIG. 3 schematically shows further details of the embodiment of FIG. 1, in particular with regard to the arrangement of the object 1 relative to the lens 13 (connected with the laser source 11) and relative to the detector device 20. This design is particularly adapted for applications wherein the object 1 is an animal, like a mouse, and the ROI is a tissue portion or an organ within the animal. The frequency domain optoacoustic tomographic imaging apparatus 100 includes the source device 10, the detector device 20, the image data acquisition and processing device 30, and the control device 40 as described above.

The carrier device 50 comprises a platform 51 which is arranged in a container unit 52 (imaging tank). The container unit 52 is filled with a coupling medium 53, e. g. a matching fluid like water, gel or oil. The platform 51 includes an object holder fixing the object 1 in a specific position for imaging. The size of the object 1 and the container unit 52 is shown for illustrating purposes and can be adjusted to different objects with different geometries and dimensions.

The detector element 21 of the detector device 20 is mounted on the rotation stage 61 of the motion device 60 with the light guiding optical fiber 12 and lens 13. A tomographic data set can be acquired by rotating the detector element 21 and the light guiding optical fiber 12 with the lens 13 simultaneously around the object 1, ending up in a volumetric data set for elevation motion of both the detector element 21 and the light guiding optical fiber 12 with the lens 13.

The number of detection and illumination elements is not limited to one unit as shown in the embodiments of FIGS. 1 and 3. In particular, an array of continuously emitting sources and/or a detector array can be provided. With a detector array comprising a plurality of detector element distributed around the object, the rotation stage 61 can be omitted. The illumination is furthermore not limited to an optical fiber but can be carried out with a free beam and optical mirrors, guiding the light beam onto the object without optical fibers.

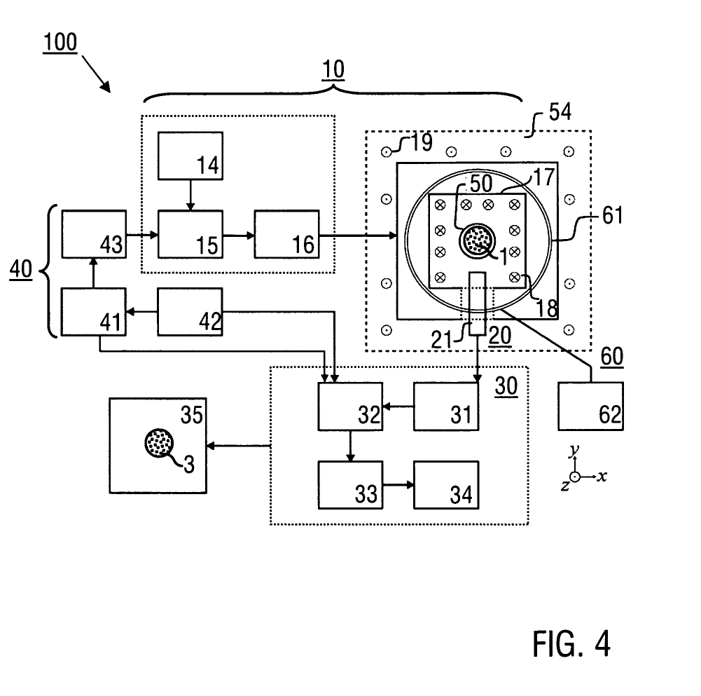

FIG. 4 shows a schematic drawing of a preferred embodiment of a frequency domain thermo-acoustic tomographic imaging apparatus 100 with its main components source device 10, detector device 20, image data acquisition and processing device 30, control device 40, carrier device 50 accommodating the object 1, and motion device 60. Contrary to the frequency domain optoacoustic imaging shown in FIG. 1, the thermo-acoustic tomographic imaging apparatus 100 is based on a radiofrequency and/or magnetic excitation of the object 1. Therefore, a shielding device 54 is provided for shielding the surroundings of apparatus 100 against electromagnetic fields.

The source device 10 is a radiation device which comprises a power unit 14 supplying energy for the source device 10, a dedicated switching circuit 15 converting the low input power to high power for an excitation unit, and a matching circuit 16 adapting the excitation unit to the switching circuit 15. The excitation unit can be made of any energy radiating element, however with respect to the magnetic focusing ability of coils, the excitation unit is preferably implemented as an electromagnetic coil 17. The free space magnetic field lines inside (18) the coil 17 and outside (19) the coil 17 demonstrate the direction of the electromagnetic field and the focusing capabilities. An imaging plane is spanned perpendicular to the magnetic field lines. It is to be noted that the direction of the magnetic field lines as well as the geometry and dimension of the excitation unit are depicted by way of an example and for the purpose of illustrative demonstration of the basic concept. Furthermore, the number of excitation elements is not limited to one as depicted in FIG. 4, however, the number of excitation units is only limited by the available space.

Acoustic pressure signals are detected with the detector device 20 which is sensitive to mechanical pressure waves induced by electromagnetic absorption. The detector device 20 is comprised of at least one detector element 21 but preferably of multiple elements, e.g. a phased detector array. For the frequency domain thermo-acoustic imaging, the detector device 20 is based e. g. on a PZT/PVDF transducer but is preferably based on an optical detector like an optical interferometric mechanical stress detector. The detector device 20 is chosen according to the size of the object 1 and the excitation frequency of the excitation.

As with the embodiment of FIG. 1, the control device 40 includes the signal generator 41 and the trigger generator 42 operating as a synchronization device. Additionally, the control device 40 includes a switch control unit 43 which drives the switching circuit 15. The signal generator 41 provides the waveform modulation of the coil 17. Preferably, the excitation signal (stimulation signal) is frequency modulated in a low frequency (LF−f=30−300 kHz), medium frequency (MF−f=0.3-3 MHz) or high frequency band (HF−f=3−30 MHz), but can also be of lower frequency (f<30 kHz) or higher frequency (f>30 MHz).

The image data acquisition and processing device 30 includes an amplifying unit 31 (optionally provided) which is used for pre-amplification of the induced CW thermo-acoustic signals, a data acquisition device 32, a signal processing and storing unit 33 and an image reconstruction unit 34 which is connected with an output device 35, e. g. displaying the reconstructed image 3 which represents an equivalent map of electromagnetic absorption within the ROI. The output device 35 can be the screen of a computer, a printer or a data storage device.

The data acquisition device 32 is detecting the thermo-acoustic signals as induced by the coil 17, the reference signal originating from the signal generator 41 and is synchronized by the trigger generator 42. The raw signals can further be processed and stored by the signal processing and storing unit 33. The signal processing and storing unit 33 performs the correlation processing between the thermo-acoustic signal and the reference signal and optionally fulfills spectral filtering tasks. The image 3 is reconstructed in the image reconstruction unit 34 and displayed on the computer screen 35.

A tomographic data set for cross sectional views of the object 1 is acquired by multiple projections around the object 1. In the embodiment of FIG. 4, the object 1 is rotated with a rotation stage 61 parallel to the imaging plane, wherein the rotation stage 61 is controlled by a motion controller 62. It is to be noted that volumetric images of the target can be generated by translating either the object 1 in the imaging plane or moving the acoustic detector element(s) relative to the object 1 in the elevation axis perpendicular to the imaging plane.

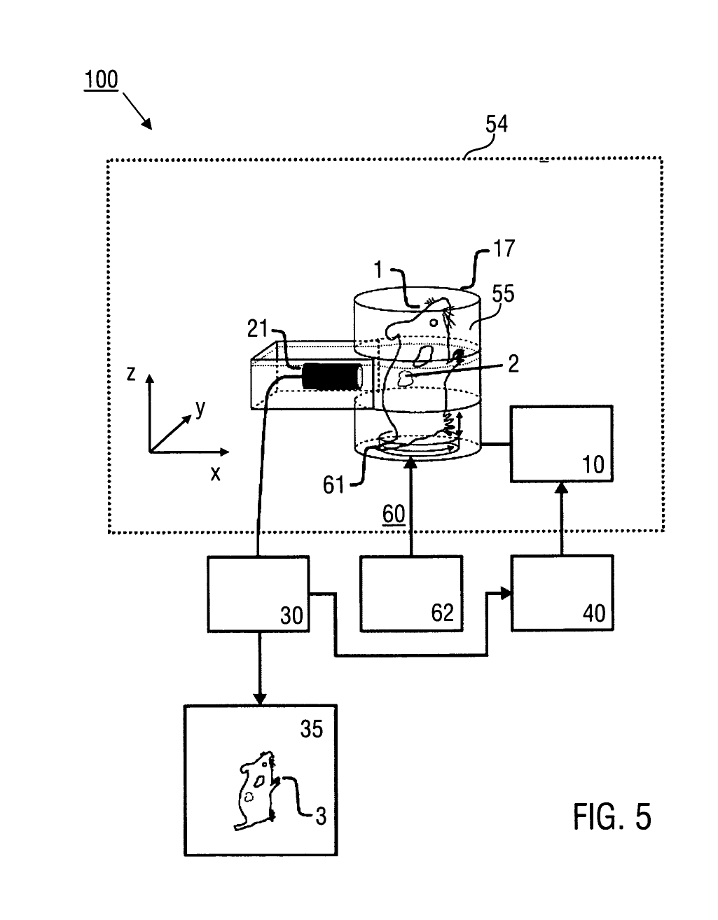

FIG. 5 shows an example of an experimental setup for a frequency domain thermoacoustic tomography scanner according to the invention, for the preferred application of small animal imaging. The frequency domain thermo-acoustic imaging apparatus 100 is composed of the source device 10 with the object 1 being placed within the excitation unit 17 (e.g. an electromagnetic coil), the detector device 20, the acquisition and processing device 30, the control unit 40 and the motion device 60 with the motion stage 61. Furthermore, the apparatus 100 is optionally placed within a shielding unit 54 which covers the coil 17, the object 1, the source device 10, the detector device 20 and the motion stage 61. The control device 40 and the acquisition and processing unit 30 are placed outside the shielding unit 54 which can be comprised of a chamber made of a copper grid or a nickel alloy foil, such as a mu metal foil or any other material absorbing or reflecting magnetic fields or electric fields (electromagnetic fields). Preferably, a matching fluid 55, such as water, oil or gel, is filled between the coil 17 and the object 1. In the illustrated implementation, the motion stage 61 rotates the object for 360° data acquisition and translates the object on the z-axis for volumetric data, however, with minor changes to the setup a tomographic data set can also be acquired by rotating and translating the acoustic detector element 21.

As an example, the object 1 comprises a mouse, wherein the ROI 2 is e. g. a subcutaneous tumor. The mouse is placed within the coil 17. For imaging the ROI 2, the mouse is continuously excited with the electromagnetic energy input emitted by the coil 17, and the acoustic wave detector element 21 is continuously detecting induced pressure waves from the ROI 2. A tomographic data set is either collected by rotating and translating the mouse or by moving the detector element 21.

The frequency domain thermo-acoustic system of FIG. 5 can be used for combined biomedical imaging and therapeutic treatment of small animals and tissue at a mescoscopic scale. Applied contrast agents can simultaneously operate as marker substances and therapeutic agents for a hybrid imaging and therapeutic system (see e. g. US 2009/0081122 A1, U.S. Pat. No. 5,411,730). A preferred method would include administration of contrast agents featuring RF/magnetic absorption, imaging at a power level sufficient for the tomography imaging and dynamically monitoring the distribution of contrast agents throughout the tissue, increasing the power level for thermal ablation once the agents are accumulated in the desired region, e. g. the tumor, and imaging at reduced power level after treatment of localized regions. Monitoring the distribution of contrast agents can be done with the output device 35. The process of thermal ablation can also be imaged in real time, providing insight in the progress of thermal therapy.

### 3. Further Applications

The invention can be utilized for various applications especially in the biomedical field, although not limited to medical and biological imaging and therapy. In the following, possible applications related to structural and functional/molecular imaging of biological tissue are described. It is to be noted that all applications listed below are not limited to small animals but can also be applied to humans.

The FD thermo-acoustic tomography imaging apparatus of the invention can be applied at a microscopic, mesoscopic and macroscopic scale. Implementation of the imaging apparatus is not limited to the size of the target; however, the imaging apparatus can be used for biological tissue imaging like small and big animal imaging, human imaging, plant imaging but also for non-biological imaging like industrial component imaging (e.g. nondestructive testing, material deficiencies), food and drink screening, soil or geological imaging. The imaging apparatus is capable of imaging only small parts of a human or mouse, but can also be used for whole human and animal imaging. Furthermore, imaging can be performed ex-vivo, in-vivo and in-vitro.

In particular with respect to the microscopic imaging scale, the FD thermo-acoustic tomography imaging apparatus can be used for imaging resolutions <100 μm for screening of tumor and cancer vascularization and also for imaging at a cellular and subcellular level. At this scale, the imaging apparatus can be applied for single red blood cell imaging, screening oxygen release from hemoglobin.

In general, the FD thermo-acoustic tomography imaging apparatus can be used for biomedical applications like disease screening both in animals and humans, such as tumor and cancer imaging. Moreover, further applications include screening biological tissue disorders like inflammation processes, vascularization of biological tissue in combination with imaging of anomalies in tissue vasculature, neurological diseases and metabolic diseases. Further applications include tissue growth monitoring, physiological imaging of biological tissue, neurological imaging and cardiovascular imaging.

Another important application includes blood imaging across the whole electromagnetic spectrum. In the optical regime, the system can resolve hematologic diseases since blood in its oxygenated and deoxygenated state has different absorption characteristics. In the RF/microwave region, the imaging apparatus can be applied for screening of iron content in hemoglobin, imaging e.g. iron deficiencies in blood cells.

Of particular interest, especially in combination with optical biomarkers, is the optical excitation of targets and extrinsically administered biomarkers by means of multispectral illumination at several wavelengths. Biomarkers include endogenous markers like intrinsic fluorochromes and chromophores (e.g. fluorescent proteins) and exogeneous agents like fluorescent dyes, fluorochromes, carbon based (nano-, micro-)particles, (nano-, micro-) particles based on noble (e.g. gold, silver) or other metal like (nano-, micro-)particles, fluorescent proteins, fluorescent conjugates and chromophoric markers. In a multispectral scenario, optical illumination of biological tissue at multiple wavelengths allows for correction of intrinsic optical contrast, originating from tissue chromophores such as blood (oxy- and deoxy-hemoglobin), melanin or fat. Thus, the distribution of biomarkers within the tissue can be resolved, suppressing the optical contrast of biological tissue. Generally speaking, the multispectral approach is not limited to the optical regime but can also be implemented for RF/magnetic/microwave excitation, applying contrast agents which feature RF/magnetic/microwave absorption such as conductive (nano-,micro)particles like carbon (nano-,micro)particles, particles based on noble (e.g. gold and silver) and other metals or magnetic particles (ferro, ferri, para, dia, superparamagnetic).

The imaging apparatus can also be implemented as a real time scanner with multiple detectors (e.g. a detector array) and one or multiple illumination patterns. Thus, changes in biological tissue like blood perfusion can be recorded dynamically; moreover, specific regions or organs of the human or animal body can be screened on a dynamical basis, either by monitoring hemoglobin or extrinsically administered contrast agents (like e.g. kidney perfusion imaging with contrast enhancers). The excitation wavelength is not limited to the optical regime but covers the whole electromagnetic spectrum, with application of optical contrast agents for optical excitation (as listed above) and RF/magnetic/microwave contrast agents for RF/magnetic/microwave excitation.

The imaging apparatus can furthermore be applied for structural and functional imaging of certain organs of the human or animal body like the liver which features high iron content.

### 4. Experimental Imaging Results

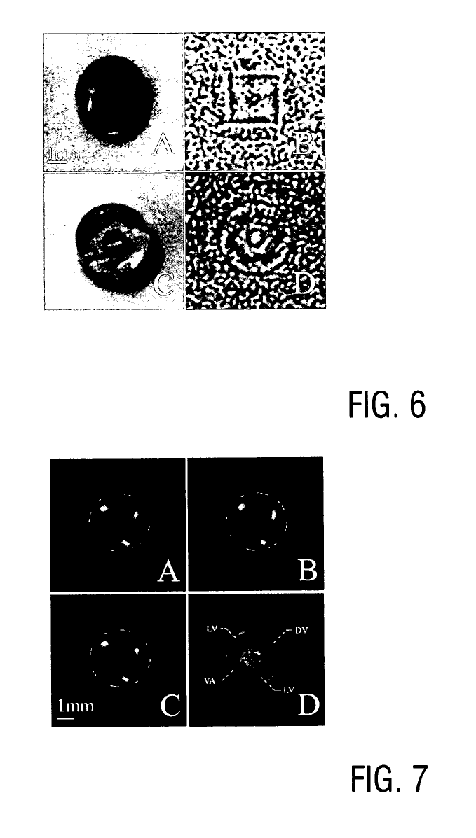

FIG. 6 shows a tomographic data set acquired from two agar phantoms with defined optical absorption inclusions in different geometries. FIG. 6A depicts the photograph of the circular shaped agar phantom with the rectangular shaped agar inclusion. The optical absorbing inclusion was made of agar mixed with India Ink, yielding an optical absorption coefficient of 2 cm⁻¹. The corresponding tomographic FD reconstruction after correlating the modulation signal with the optically induced acoustic signal and projecting the processed data back on a predefined virtual grid is showcased in FIG. 6B, revealing size and shape congruence to the photograph of FIG. 6A. Similarly, FIG. 6C shows a photograph of a second circular shaped agar phantom with a ˜1 mm mixed agar India Ink inclusion, exhibiting an optical absorption coefficient of 2 cm⁻¹. The corresponding FD cross sectional reconstruction is depicted in FIG. 6D, demonstrating the layout of the phantom with the small insertion of absorbing agar surrounded by the outer layer of agar.

Referring to FIG. 7, a tomographic data set was acquired in-vivo from a mouse tail. The measurement protocol consisted of mouse gas anesthesia (isoflurane) followed by catheterization of the right vein at approximately 2 cm from the distal end with the mouse attached to a custom made tail holder. At first, the mouse tail was imaged without contrast enhancement at a height of ˜4 cm from the distal end. After the first FD tomographic measurement, 130 nmol of Indocyanine Green (ICG) was injected via the catheter in the mouse tail and a second measurement was immediately thereafter initiated. To compensate for the ICG clearance from the blood stream though the hepatobiliary tract, occurring during the acquisition time of ˜10 min/image, an additional 100 nmol of ICG was administered at projection angle 140°. Subsequently to the second, post-ICG tomographic experiment, a third FD tomographic data set was obtained approximately 10 min from the initial ICG injection to monitor the ability of the FD optoacoustic tomography system to record changes in response to ICG clearance from the blood circulation system of the mouse dynamically. After the in-vivo measurements, the mouse was euthanized and prepared for cryoslicing, thus freezing the mouse to −80° C. and cryoslicing the mouse tail. At the height of the FD optoacoustic tomography measurements, photographs were taken for comparison with the FD optoacoustic tomography reconstructions. FIG. 7A highlights the tail blood vessels such as the dorsal vein (DV), the lateral caudal veins (LV) and the ventral caudal artery (VA) (see FIG. 7A and FIG. 7D). FIG. 7B illustrates the absorption increase following ICG injection and showcases an optical absorption gain of approximately a factor of 2. FIG. 7C depicts the FD tomographic reconstruction of the mouse tail approximately after 10 min from the initial ICG injection, unfolding a contrast decrease at a scale which is as expected between the maximum observed on FIG. 7B and the baseline of FIG. 7A.

The features of the invention disclosed in the above description, the figures and the claims can be equally significant for realizing the invention in its different embodiments, either individually or in combination.

1. An imaging apparatus, configured for thermoacoustic tomographic imaging a region of interest in an object, comprising:
a source device being arranged for emitting an electromagnetic energy input into the region of interest,
a detector device being arranged for detecting mechanical wave response signals generated in the region of interest along multiple angular projection directions in response to the electromagnetic energy input, and
an image data acquisition and processing device being arranged for providing tomographic image data representing the image of the region of interest on the basis of the mechanical wave response signals,
wherein the source device is adapted for continuously emitting the electromagnetic energy input with a predetermined input modulation, and the image data acquisition and processing device is adapted for converting the mechanical wave response signals into the frequency domain and for performing data processing and image reconstruction in the frequency domain or in the time domain.

2. The imaging apparatus according to claim 1, wherein the source device is adapted for continuously emitting the electromagnetic energy input with the input modulation including at least one of frequency modulation, chirp modulation, amplitude modulation, phase modulation or digital modulation.

3. The imaging apparatus according to claim 2, wherein the input modulation includes at least one of a linear, logarithmic, sin-like, square-like, or triangle-like frequency modulation.

4. The imaging apparatus according to claim 1, wherein the detector device includes at least one of the following:
at least one acoustic detector element being movable relative to the object,
a detector array including multiple acoustic detector elements being fixedly arranged around the object, or
an optical or interferometric device.

5. The imaging apparatus according to claim 1, wherein the source device includes an array of continuously emitting sources.

6. The imaging apparatus according to claim 5, wherein the sources of the array are adapted for emitting the electromagnetic energy input with different wavelengths.

7. The imaging apparatus according to claim 1, wherein the source device is adapted for continuously emitting the electromagnetic energy input in an optical wavelength range including at least one of UV, VIS or IR wavelength ranges.

8. The imaging apparatus according to claim 7, including at least one of the following features:
the source device comprises at least one of an amplitude modulated CW laser or an amplitude modulated light emitting diode, or
the source device is provided with at least one of an acousto-optic modulator, electro-optic modulator, a mechanical chopper or an electrically modulated power source.

9. The imaging apparatus according to claim 1, wherein the source device is adapted for continuously emitting the electromagnetic energy input in a radiofrequency range.

10. The imaging apparatus according to claim 9, including at least one of the following features:
the source device comprises at least one radiofrequency source emitting in the low MHz region, or
the source device comprises an energy coupling element.

11. The imaging apparatus according to claim 1, including a reconstruction unit processing the data and reconstructing a tomographic image of a distribution of electromagnetic energy absorbers within the region of interest.

12. The imaging apparatus according to claim 1, further including a carrier device being arranged for accommodating the object, wherein the carrier device is configured for moving the object relative to the detector device.

13. An imaging method for thermoacoustic tomographic imaging a region of interest in an object, comprising the steps of:
emitting an electromagnetic energy input into the region of interest with a source device,
detecting mechanical wave response signals generated in the region of interest along multiple projection directions in response to the electromagnetic energy input with a detector device, and
providing tomographic image data representing the image of the region of interest on the basis of the mechanical wave response signals originating from electromagnetic energy absorption with an image processing device,
wherein the source device is continuously emitting the electromagnetic energy input with a predetermined input modulation, and the image processing device is converting the mechanical wave response signals into the frequency domain and for performing data processing and image reconstruction in the frequency domain or in the time domain.

14-20. (canceled)

21. The imaging method according to claim 13, wherein the object includes at least one of biological tissue, biomedical material or industrial material.

22. The imaging method according to claim 13, wherein the object includes a distribution of marker substances including at least one of a biomarker or a radiofrequency absorber.

23. The imaging method according to claim 22, wherein the marker substances include at least one of fluorescent proteins, chromophoric or fluorescent molecules, particles (nano-, micro-), photodynamic therapy agents, paramagnetic particles, super-paramagnetic particles, ferromagnetic particles, diamagnetic particles, magnetic loss particles, carbon particles, ceramic particles, electrically conducting particles, particles from noble metals, semiconducting particles or activatable substrates.

24-27. (canceled)

28. The imaging method according to claim 13, including the steps of:
operating the source device in a treatment mode with an increased level of electromagnetic energy input, and
subjecting the object to a thermal treatment by the increased level electromagnetic energy input.

29. The imaging method according to claim 13, including at least one of the following steps:
at least one of the detector device, the source device or parts thereof are inserted inside a blood vessel for intravascular imaging thereof.

30. The imaging method according to claim 13, including at least one of the following steps:
at least one of the detector device, the source device or parts thereof are inserted inside a tissue cavity for catheter imaging thereof.

31. The imaging method according to claim 13, wherein at least one of the detector device, the source device or parts thereof are arranged in a hand held unit.

32. The imaging method according to claim 13, wherein the spatial distribution of absorbers in the region of interest is reconstructed in frequency domain using a pulse compression method, involving the cross correlation from an input modulation signal with the mechanical wave response signals, or a reconstructing method based on Diffraction Tomography with the Fourier Diffraction Theorem, employing wave solutions using diffracting sources and subsequently inverting a corresponding model matrix describing the geometrical and operational parameters of the illumination and detection process.
